(19) United States
(12) Patent Application Publication
(10) Pub. No.: US 2024/0091395 A1
(43) Pub. Date: Mar. 21, 2024

(54) MANUALLY GUIDED DEVICE HAVING HIGH VOLTAGE POWER ELECTRONICS

(71) Applicant: BSH Hausgeräte GmbH, München (DE)

(72) Inventors: Stefan Hassfurter, Maroldsweisach (DE); Thomas Mudra, Kastl (DE); Tobias Wende, Prien (DE); Julius Geis, Burkardroth (DE)

(21) Appl. No.: 18/255,417

(22) PCT Filed: Nov. 16, 2021
(86) PCT No.: PCT/EP2021/081791

§ 371 Date: Jun. 1, 2023

(30) Foreign Application Priority Data
Dec. 1, 2020 (DE) 10 2020 215 104.0

(51) Int. Cl. A61L 2/14; A61L 2/26

(52) U.S. Cl. CPC A61L 2/14; A61L 2/26; A61L 2202/11; A61L 2202/16; A61L 2202/26

(57) ABSTRACT

A manually guided device has an electrically insulating housing in which power electronics operated by high voltage are accommodated. The device is used in particular for treating an object, in particular by way of plasma. Increased comfort and simplified manufacturing of the device result from an electrically conductive equipotential bonding device which is electrically isolated from the power electronics and which forms, at least in part, an outer surface of the device with which a user's hand is in contact during use.

The present invention relates to a hand-guided device which is used in particular so as to treat an object and which in a housing has power electronics that are operated using high voltage.

Hand-guided devices offer in particular the advantage of a simple use. They can be used for example so as to treat an object. Generic, hand-guided devices have power electronics that are used during operation of the electrical supply, in particular the power that is required for the treatment. In this case, the power electronics are accommodated in a housing of the device. In this case, hand-guided devices during use are gripped and/or guided by at least one hand of a user. In this case, the hand of the user lies on the housing of the device. In order to counteract undesired electrical transmissions to the hand of the user, the housings are usually electrically insulating.

If the power electronics are operated with high voltage, it is possible during use of the device despite the electrically insulating housing for energy to be transmitted to the hand and consequently for an electrical discharge and/or so-called back discharges to occur via the hand of the user. The probability and frequency of such discharges and/or back discharges, referred to hereafter jointly as discharges, increases if the power electronics are additionally operated using an AC voltage, in particular high frequency AC voltage.

Aforementioned discharges occur in particular in the case of such hand-guided devices in which the device cannot be entirely outwardly electrically insulated for the intended purpose.

Examples of such hand-guided devices are devices that generate plasma via a plasma source that is accommodated in the housing and is supplied electrically using the power electronics, wherein the plasma must exit the housing in order to treat an object. Such hand-guided devices that generate plasma during operation are known for example from DE 10 2018 213 143 A1, DE 10 2018 2013 144 A1, CN 2 05 814 739 U and also CN 1 08 771 767 A.

Although such devices are designed in such a manner that the discharges are harmless. The discharges are nevertheless perceived as unpleasant by the user.

The present invention is therefore concerned with the object of disclosing an improved or at least different embodiment for a hand-guided device of the type that is mentioned in the introduction, which is characterized in particular by an improved comfort for a user while simultaneously being easier to produce.

This object is achieved in accordance with the invention by the subject matter of the independent claim 1. Advantageous embodiments are the subject matter of the dependent claims.

The present invention is based on the general idea in the case of a hand-guided device, which has power electronics that are operated in an electrically insulating housing with high voltage, to design an outer surface, which during use is in contact with a hand of a user, as at least in part electrically conductive. As a consequence, a possible surface for the electrical potential equalization with the hand is enlarged. This results in a transmission of energy to the hand, which occurs due to the high voltage and consequently, in the event of a discharge and/or so-called back discharges via the hand of the user, a reduction of the current density. The reduction of the current density during the aforementioned transmission of energy in this case leads to the fact that the noticeability for the user is reduced, in particular is reduced in such a manner that the user does not notice such discharges and/or back discharges, hereafter referred to jointly as discharges. Consequently, with unchanged power electronics, in particular electrical voltage supply, the comfort for the user is increased. Simultaneously, this increase in comfort can be implemented in a simple and cost-effective manner.

According to the inventive idea, the hand-guided device has the housing, which is electrically insulating and in which the power electronics that are operated with high voltage are accommodated. The hand-guided device is used in particular so as to treat an object. In this case, the power electronics are used for the electrical supply so as to generate and/or provide the energy that is required for the treatment. The device is designed in such a manner that during use, a user grips the housing at least in regions. In accordance with the invention, the device has an electrically conductive potential equalizing facility that at least in part forms an outer surface of the device, which during use is in contact with the hand of the user. The potential equalizing facility in this case is electrically isolated from the power electronics. The potential equalizing facility accordingly leads to the enlargement of the surface that is provided during the aforementioned discharges, which is hereafter also referred to as a potential equalizing surface. The potential equalizing surface is thus enlarged over the outer surface using the potential equalizing facility, with the result that the current density to the user, which occurs in the event of discharges, is reduced.

A hand-guided device or device that can be hand-guided in the present case is to be understood to mean a mobile device that is moved by hand by a user—in other words by means of one or both of their hands—during operation of the device over the object that is to be treated, in particular over a surface of the object which is to be treated. Typically, such a hand-guided device in contrast to stationary devices can be portable for the user and the device is adapted to this portability with regard to its mass and dimensions. This leads to a further improved comfort of the device.

“Electrically isolated” in the present case is in particular to be understood to mean that an electrical connection does not exist and/or that possible mechanical connections are electrically insulating. This means in particular that an electrical connection does not exist between the potential equalizing facility and the power electronics. This also means in particular that possible mechanical connections between the potential equalizing facility and the power electronics are electrically insulating.

In principle, the power electronics can be operated using a DC voltage.

It is conceivable and preferred to operate the power electronics using an AC voltage, in particular a high frequency AC voltage. This means that the power electronics are operated in particular using a high frequency high voltage. High voltage in the present case is to be understood to mean, in the case of a DC voltage, voltage values of more than 1 kV and, in the case of AC voltages, voltage values of more than 1.5 kV.

High frequency is to be understood to mean in particular voltage frequencies of more than 1 kHz. It is preferred that the power electronics are operated using a frequency of multiple 10 kHz, in particular 100 kHz.

In principle, the hand-guided device can be used for an arbitrary treatment of the object.

In particular, plasma is used for the treatment of the object, in particular a surface of the object. Accordingly, the device has a plasma source that is accommodated in the housing and is supplied electrically using the power electronics so as to generate the plasma. It is preferred that the plasma source is designed in such a manner that it generates cold plasma during operation. The device can consequently be implemented in a compact manner and/or with a reduced energy consumption. Moreover, cold plasma can be used effectively and with at least a reduced thermal interaction for the treatment of the object. In this manner, the risk of damage, which is caused thermally to the object that is to be treated, and/or an unpleasant sensation of the user, which is caused thermally, are reduced. As a consequence, in this manner the comfort is further improved. The plasma source and the potential equalizing facility are electrically isolated in an advantageous manner.

Object in the present case is to be understood as any biological and non-biological object that can be treated using the device, preferably using the plasma that is generated by means of the plasma source. During the treatment, there is an interaction between the plasma and the object and/or elements that are present on the object, in particular odor elements, which leads to a corresponding change. Object is to be understood to mean in particular textile objects such as items of clothing. Objects are also such objects that have a textile surface, which are to be treated using the plasma, such as for example furniture, mattresses and the like. Textile materials are for example natural, plant-based such as natural fibers of an animal origin such as cotton, sheep's wool, silk, linen, felt; textile materials are also synthetic clothing materials having chemical fibers such as for example nylon. Furthermore, the object can have, at least on the surface, ceramic, plastic, feathers, leather, glass, wood, metal or mixtures thereof.

During the interaction of the plasma with the object, in particular the surface, in particular odor elements are inactivated, in other words destroyed and/or changed in such a manner that the perceived odor disappears and/or is changed. Odor elements are in general organic compounds or include such compounds. Odor elements include for example cell walls, bacteria, germs, viruses, fungi and the like. During the interaction with the plasma, the odor elements can be negatively charged owing to the bombardment with the electrons that are present in the plasma. Owing to the electrostatic repulsion, this can lead to mechanical stresses up to exceeding the tensile strength and an accompanying destruction of the odor molecules. Low pressure plasmas or cold plasmas are in this case particularly well suited for inactivating odors on textiles and/or normal household surfaces and the like since a thermal damage of such a surface does not occur or is at least reduced.

In the case of preferred embodiments, the plasma source has a circuit board. In particular, the plasma source is based on at least one circuit board. The circuit board in this case can be part of the power electronics. In the case of such an embodiment of the plasma source, it is possible to implement the potential equalizing facility in a simplified and compact manner. As a consequence, the comfort is increased due to the compactness. The simplified implementation moreover leads to a simple manufacturing of the device.

The housing can be designed in such a manner that the plasma that is generated using the plasma source exits the device via an outlet surface, in particular an outlet opening, of the housing in order to interact with the object so as to treat the object.

Embodiments are preferred in which the housing and the plasma source are designed in such a manner that the plasma source generates the plasma outside the housing on a treatment surface. For this purpose, the plasma source can have on the treatment surface a protruding, preferably elongated electrode that is surrounded by a ceramic plate. In an advantageous manner, the potential equalizing facility is electrically isolated from the treatment surface.

It is preferred that the potential equalizing facility is spaced with respect to the outlet surface. Consequently, in particular a change of the generated plasma due to the potential equalizing facility is prevented or at least the risk of the change is reduced. Moreover, electrical transmissions of the plasma to the potential equalizing facility and as a consequence via the potential equalizing facility to the user are prevented or the corresponding risk is at least reduced. This leads to an improved usability of the device and also to an increased comfort. It is preferred that the potential equalizing facility is electrically isolated from the outlet surface.

The potential equalizing facility in the case of the preferred embodiments has an electrically conductive body that surrounds the power electronics and is spaced with respect to these power electronics. The body, hereafter also referred to as annular body, thus surrounds the power electronics and simultaneously at least in part forms the outer surface. Consequently, a simple and effective enlargement of the potential equalizing surface and consequently a simple and effective extension of the current density that occurs in the event of discharges is created.

The housing can have an upper part and a lower part that in each case can be designed as a shell. The upper part and the lower part in this case delimit an interior space of the housing in which the power electronics, in particular moreover the plasma source, are accommodated.

In the case of preferred embodiments, the annular body is arranged between the upper part and the lower part and is arranged on the outer side of the housing. This leads to a simple embodiment of the device and simultaneously to a simple enlargement of the potential equalizing surface. Furthermore, the annular body can moreover function in this manner as a seal. Consequently, the production of the device is further simplified.

In lieu of the annular body or in addition to the annular body, the potential equalizing facility can have at least one strip that runs longitudinally, is electrically conductive and is spaced with respect to the power electronics and in part forms the outer surface. It is conceivable in particular to provide a potential equalizing facility with two such strips, wherein the strips are arranged lying opposite.

Alternatively or in addition thereto, the potential equalizing facility can have a layer that is at least in part applied to the housing and is electrically conductive, wherein the layer at least in part forms the outer surface of the device. It is consequently possible to implement the potential equalizing facility in a simplified manner and with a particularly large potential equalizing surface. Moreover, it is possible in this manner to produce the device in a compact manner and with a reduced weight. The comfort is further improved as a consequence.

The layer of the potential equalizing facility can in principle have an arbitrary composition, provided that the layer is electrically conductive.

It is conceivable in particular that the layer features an electrically conductive plastic. In particular, the layer can be an electrically conductive plastic layer that is applied to the outer side of the housing.

Alternatively or in addition thereto, the layer can feature at least one metal and/or one metal alloy. In particular, the layer can be a metal layer and/or metal alloy layer that is applied to the outer side of the housing.

The device can have an operating element, for example a switch, for manually activating and deactivating or switching on and switching off the device. The operating element is expediently attached to the housing. In this case, the potential equalizing facility can at least in part form at least one outer surface of the actuating element. This means that at least the outer surface of the operating element can at least in part be part of the potential equalizing facility. Consequently, a simple implementation of the potential equalizing facility is realized.

It is preferred if the device is designed in such a manner that it is only activated when the operating element is actively actuated. This means that the user must permanently actuate the actuating element in order to use the device. As a result, a contact of the hand of the user with the actuating element is required. Due to the at least in part use of the actuating element as a potential equalizing facility, an enlargement of the potential equalizing surface is consequently achieved in a simple and effective manner and consequently the current density is reduced. The comfort is consequently improved in a simple and effective manner.

Further important features and advantages of the invention are provided in the subclaims, the drawings and the associated description of the figures with the aid of the drawings.

It is understood that the above-mentioned features and the features that are still to be explained below can be used not just in the respectively disclosed combination but rather also in other combinations or in isolation without departing the scope of the present invention.

Preferred exemplary embodiments of the invention are illustrated in the drawings and are further explained in the following description, wherein identical reference characters relate to identical or similar or functionally identical components.

In the drawings, in each case schematically,

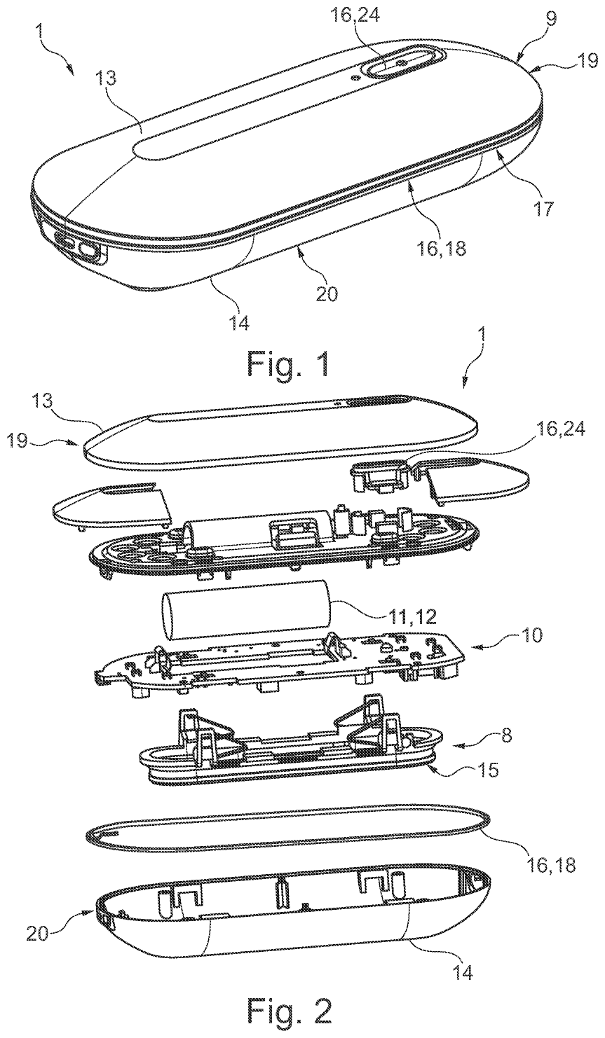

FIG. 1 shows an isometric view of a hand-guided device,

FIG. 2 shows an exploded view of the device,

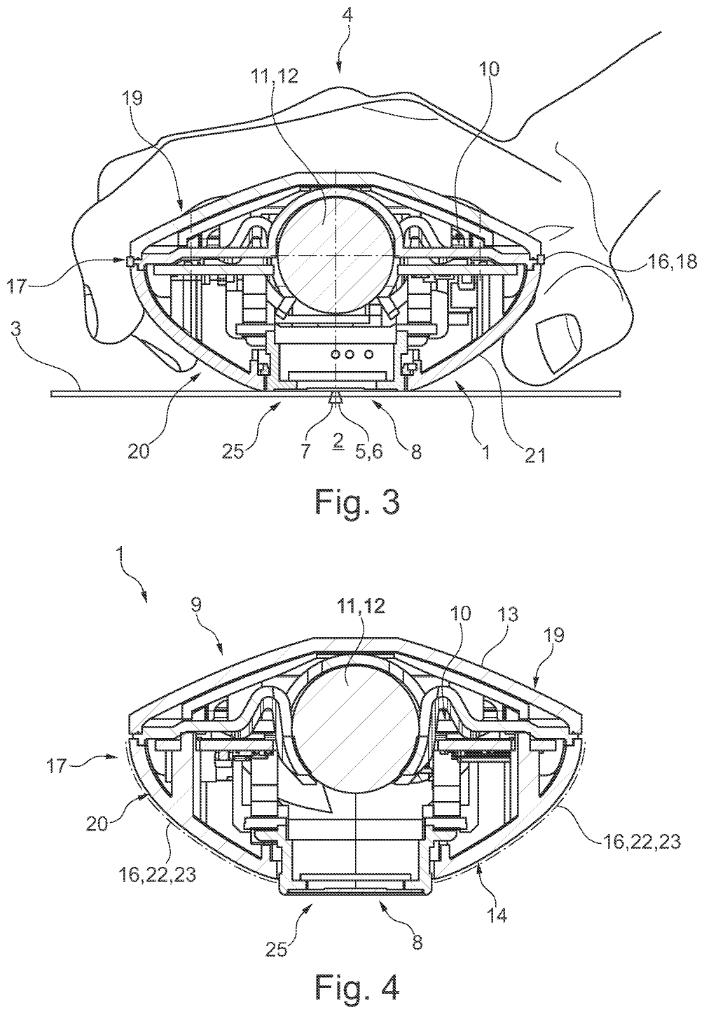

FIG. 3 shows a section through the device in use,

FIG. 4 shows a section through the device in the case of another exemplary embodiment,

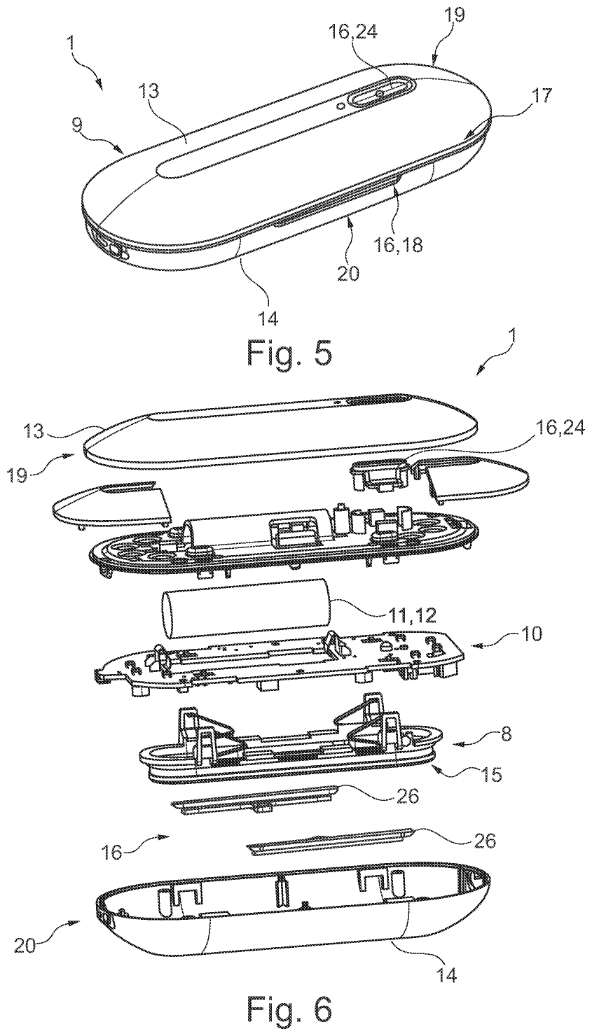

FIG. 5 shows an isometric view of the device in the case of a further exemplary embodiment,

FIG. 6 shows an exploded view of the device in FIG. 5,

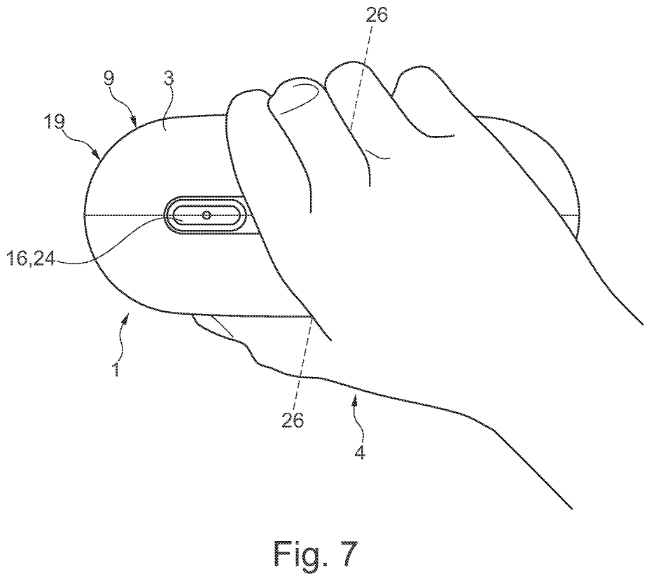

FIG. 7 shows a plan view of the device in use.

A hand-guided device 1, such as is illustrated for example in FIGS. 1 to 7, is used in particular so as to treat an object 2 (cf. FIG. 3). Corresponding to FIG. 3, the device 1 for treating the object 2 is guided and consequently moved over a surface 3 of the object 2. The hand-guided device 1 in this case, as is illustrated in FIG. 3 and in FIG. 7, is guided during use using at least one hand 4 over the object 2, in particular the surface 3. Thus, the weight and dimensions of the device 1 are such that it can be moved with one hand 4 or with two hands 4. Moreover, the device 1, as is apparent in FIG. 3 and FIG. 7, can be gripped using one hand 4.

In order to treat the object 2, in particular the surface 3, in the illustrated exemplary embodiments plasma 5 is used, in particular cold plasma 6, which is indicated in FIG. 3 by a current 7. As is apparent in FIGS. 2 to 4, the device 1 for this purpose has a plasma source 8 that is accommodated in a housing 9 of the device 1. During use, the plasma source 8 is in contact with the object 2, in particular with the surface 3. In this case, as a comparison of FIGS. 3 and 4 illustrates, the plasma source 8 that is accommodated such that it can move in the housing 9 is pushed and shifted in the direction of the housing 9. In the shifted-in state, the plasma source 8 generates the plasma 5. The housing 1 is electrically isolating, for example is made of plastic. The plasma source 8 is electrically supplied with a supply voltage via power electronics 10 that are visible in FIGS. 2 to 4 and also in FIG. 6 in order to generate the plasma 7. The power electronics 10 are electrically connected to an electrical energy storage device 11, for example a rechargeable battery 12, of the device 1, wherein the energy storage device 11 and the power electronics 10 are accommodated in the housing 9. The housing 9 has an upper side 13 and a lower side 14 that is remote from the upper side 13, wherein the plasma source 8 during operation generates the plasma 5 and consequently the plasma flow 7 on the lower side 14 of the housing 9, wherein the plasma 5 interacts with the object 2 or the surface 3.

The surface 3 is for example a surface that is made from textile. In particular, the object 2 is a textile object 2. During the interaction of the plasma 5 with the object 2, in particular the surface 3, in this case odor elements are preferably inactivated.

The plasma source 8 in the illustrated exemplary embodiments has a circuit board 15, in particular is based on a circuit board 15, wherein the circuit board 15 can be part of the power electronics 10.

The power electronics 10 are operated using a high voltage, preferably a high frequency high voltage. In particular, the power electronics 10 are operated using multiple ten kHz, in particular 100 kHz. In this case, it is possible during operation for energy to be transmitted from the power electronics 10 and/or the plasma source 8 to the hand 4 and consequently for electrical discharges and/or back discharges to occur, which hereafter are also jointly referred to as discharges. Discharges of this type are perceived as unpleasant by the user. In order to eliminate or to at least reduce the unpleasant sensation, the device 1 has a potential equalizing facility 16. The potential equalizing facility 16 is electrically isolated from the power electronics 10, in particular spaced with respect to the power electronics 10 and the plasma source 8. The potential equalizing facility 16 is moreover electrically conductive and forms an outer surface 17 of the device 1 and during use the user is at least in part in contact with the outer surface using the hand 4. An enlargement of the surface that is available for the potential equalization in the event of the discharges is consequently provided with the result that the electrical current density in the event of the discharges between the hand 4 and the device 1 is reduced. In this manner, the perception and as a consequence the sensation by the user of the discharges is reduced. In particular, in this manner the discharges are no longer perceived by the user.

In the case of the exemplary embodiment that is illustrated in FIGS. 1 to 3, the potential equalizing facility 16 has an electrically conductive annular body 18 that surrounds the power electronics 10 and is spaced with respect to these power electronics. The annular body 18 in the illustrated exemplary embodiments is designed as closed. In this case, the annular body 18 with its side that is remote from the power electronics 10 forms the outer surface 17 of the device 1 that is gripped by hand 4, at least in part as is apparent in particular in FIG. 3.

In the illustrated exemplary embodiments, the housing 9 has a shell-shaped part 19, which points toward the upper side 13, hereafter referred to as the upper part 19, and also a shell-shaped part 20, which points toward the lower side 14, hereafter also referred to as the lower part 20. The upper part 19 and lower part 20 in this case delimit an interior space 21 in the housing 9 in which the power electronics 10 are accommodated. In the illustrated exemplary embodiments, moreover the plasma source 8 and the energy storage device 11 are accommodated in the interior space 21. The plasma source 8 protrudes on the lower side 11 out the housing 9 and has a treatment surface 25 on the side that is remote from the interior space 21 and the plasma 5 is generated on the treatment surface.

In the exemplary embodiment of FIGS. 1 to 3, the annular body 18 is arranged between the upper part 19 and the lower part 20. In this manner, the annular body 18 moreover fulfils a sealing function.

In the case of the exemplary embodiment that is illustrated in FIG. 4, the potential equalizing facility 16 in contrast to the exemplary embodiment that is illustrated in FIGS. 1 to 3 does not have an annular body 18. The potential equalizing facility 16 conversely has an electrically conductive layer 22 that is illustrated in FIG. 4 as dashed and is applied at least in part to the outside of the housing 9. In the illustrated exemplary embodiment, the layer 22 in this case in a purely exemplary manner is applied to the lower part 20 and consequently also to the lower side 14 of the housing 9. The electrically conductive layer 22 has an electrically conductive plastic and/or metal and/or metal alloy. In particular, the layer 22 is an electrically conductive plastic layer 23.

FIGS. 5 to 7 illustrate a further exemplary embodiment of the device 1. This exemplary embodiment differs from the exemplary embodiment, which is illustrated in FIGS. 1 to 3, by virtue of the fact that the potential equalizing facility 16 in lieu of the annular body 18 has two electrically conductive strips 26 that are spaced with respect to one another and lie opposite one another. The strips 26 are spaced with respect to the power electronics 10 and in each case the strips form the outer side 17 of the device 1 with their side that is remote from the power electronics 10 and the device is in part gripped by hand 4. In this case, the respective strip 26 runs longitudinally along a longitudinal side of the housing 9. Moreover, the respective strip 26 in the illustrated exemplary embodiment is arranged between the upper part 19 and the lower part 20. As is apparent in particular in FIG. 7, at least one of the strips 26, advantageously the respective strip 26, during use is in contact with the hand 4 and is covered by the hand 4. As a consequence, the strips 26 are not visible in FIG. 7.

The respective potential equalizing facility 16 can alternatively or in addition be realized on an operating element 24 of the device 1, which is visible in particular in FIGS. 1 and 5. The operating element 24 is used for the activation and deactivation of the device 1. In this case, the operating element 24 in the illustrated exemplary embodiments is provided on the upper side 13 of the device 1. In this case, the potential equalizing facility 16 at least in part forms a surface of the operating element 24, which is in contact with the hand 4 for the actuation of the operating element 24. This means that the operating element 24 at least on the surface, which in part forms an outer surface 17 of the device 1, can be part of the potential equalizing facility 16. In this case, it is preferred if the device 1 is only activated when the operating element 1 is actuated by hand 4, in other words during an actuation of the operating element 24. Consequently, the aforementioned discharges can only then occur if the hand 4 is in contact with the operating element 24. Since the potential equalizing facility 16 is realized on the operating element 24, an enlargement of the potential equalizing surface inevitably occurs as a consequence.

### LIST OF REFERENCE CHARACTERS

1 Hand-guided device
2 Object
3 Surface
4 Hand
5 Plasma
6 Cold plasma
7 Plasma flow
8 Plasma source
9 Housing
10 Power electronics
11 Energy storage device
12 Rechargeable battery
13 Upper side
14 Lower side
15 Circuit board
16 Potential equalizing facility
17 Outer surface
18 Annular body
19 Upper part
20 Lower part
21 Inner volume
22 Layer
23 Plastic layer
24 Operating element
25 Treatment surface
26 Strip

1-10. (canceled)

11. A hand-guided device, comprising:
an electrically insulating housing to be gripped by a hand of a user during a use of the device;
power electronics accommodated in said housing, said power electronics being operated at high voltage;
an electrically conductive potential equalizing facility that is electrically isolated from said power electronics, said potential equalizing facility being configured, during the use of the device, to form at least in part an outer surface of the device which is in contact with the hand of the user.

12. The device according to claim 11 configured for treating a textile object.

13. The device according to claim 11, further comprising a plasma source disposed in said housing, said plasma source being supplied by said power electronics and generating plasma during an operation of the device and wherein the plasma interacts with an object to thereby treat the object.

14. The device according to claim 13, wherein said plasma source is configured to generate cold plasma.

15. A hand-guided device, comprising:
an electrically insulating housing;
a plasma source accommodated in said housing and electrically supplied using power electronics, which are operated with high voltage; and
an outer surface of the device which, during use of the device, is in contact with a hand of a user and is at least in part electrically conductive.

16. The device according to claim 15, wherein said plasma source comprises a circuit board.

17. The device according to claim 15, wherein said plasma source, during an operation of the device generates plasma for the treatment of the object on an outer treatment surface, and wherein said potential equalizing facility is spaced apart from the treatment surface.

18. The device according to claim 15, wherein said potential equalizing facility has an annular body that surrounds said power electronics and in part forms the outer surface.

19. The device according to claim 18, wherein said housing has an upper part and a lower part that delimit an interior space of said housing in which said power electronics are accommodated, and wherein said annular body is arranged between said upper part and said lower part.

20. The device according to claim 15, wherein said potential equalizing facility has a layer that is applied to said housing and at least in part forms the outer surface.

21. The device according to claim 20, wherein said layer comprises an electrically conductive plastic.

22. The device according to claim 20, wherein said layer comprises at least one electrically conductive material selected from the group consisting of a metal and a metal alloy.

23. The device according to claim 20, which comprises an actuating element for selectively activating or deactivating the device, and wherein said potential equalizing facility at least in part forms a surface of said actuating element.
